(19) United States
(12) Patent Application Publication
(10) Pub. No.: US 2016/0228723 A1
(43) Pub. Date: Aug. 11, 2016

(54) UNI-POLAR PULSED ELECTROMAGNETIC MEDICAL APPARATUS AND METHODS OF USE

(71) Applicants: Hossam Abdel Salam El Sayed MOHAMED, Ottawa (CA); Houda Abdul Rahman M. AL MANSOUR, Ottawa (CA)

(72) Inventors: Hossam Abdel Salam El Sayed MOHAMED, Ottawa (CA); Houda Abdul Rahman M. AL MANSOUR, Ottawa (CA)

(21) Appl. No.: 15/024,606

(22) PCT Filed: Oct. 30, 2013
(86) PCT No.: PCT/IB2013/002441

§ 371 Date: Mar. 24, 2016

(51) Int. Cl. A61N 2/02; A61N 2/00

(52) U.S. Cl. CPC A61N 2/02; A61N 2/006; A61N 2/008

(57) ABSTRACT

Apparatus and methods for providing Uni-polar Pulsed Electromagnetic Field (UPEMF) therapy for various medical conditions. The apparatus includes a unit having an electromagnetic field producing coil and a focusing subassembly in it. The coil is arranged to be energized by associated circuitry to produce a pulsating electromagnetic field. The focusing subassembly includes sets of focusing magnets to focus the lines of flux emanating from one pole of the coil so that they are concentrated and extend for some distance generally parallel to the central longitudinal axis of the coil. A Mu metal body is provided around the other pole of the unit and around portions of the focusing magnets.

### CROSS-REFERENCE TO RELATED APPLICATIONS

N/A

### FIELD OF THE INVENTION

This invention relates generally to medical devices and methods of use and more particularly to unipolar pulsed electromagnetic medical apparatus and methods of use for treating various medical conditions.

### BACKGROUND OF THE INVENTION

The therapeutic application of Pulsed Electromagnetic Field (“PEMF”) therapy has been accepted by the Food and Drug Administration for use in humans and has become an accepted modality in treating various medical conditions. In particular, it is now used in treatment of orthopedic diseases like osteoporosis, muscle pain, arthritis, synovitis, tendinitis, neck pain, back pain and others. It is also used in treatment of aging processes of the brain, such as Alzheimer's disease, Parkinson's disease, dementia. It has also been used to treat coronary artery disease too. One of the most successful usages is for the treatments of fibro-myalgia. As is known this disease is characterized by muscle pain, sleep disturbance, generalized weakness, with some psychological elements. Typically, it has a poor response to medical treatments, except for PEMF therapy.

Typically PEMF therapy is achieved by means of apparatus making use of at least one inductive coil producing a pulsating electromagnetic field. The coil/coils are energized by applying a predetermined electrical current to one or more of the inductive coil(s) in order to produce a desirable magnetic field with specified field characteristics.

The patent literature includes disclosures of pulsed electromagnetic fields for treating medical conditions by delivering the electromagnetic radiation into the body of a patient. Examples of such prior art devices are shown in U.S. Pat. No. 5,478,303 (Foley-Nolan et al.); Pat. No. 5,518,496 (McLeod et al.); Pat. No. 6,132,362 (Tepper et al.); Pat. No. 6.179,772 (Blackwell); Pat. No. 6,261,221 (Tepper et al.); Pat. No. 6,418,345 (Tepper et al.); Pat. No. 6,524,233 (Markoll); Pat. No. 6,547,713 (Talpo); Pat. No. 6,839,595 (Tepper et al.); Pat. No. 7,175,587 (Gordon et al.); Pat. No. 8,376,925 (Dennis et al.); and in EP0048451A1 and EP1442766B1. All references cited herein are incorporated by reference.

Heretofore known methods and apparatuses which have been used for PEMF therapy while generally suitable for their intended purposes leave much to be desired from various standpoints. For example, those prior art systems which make use of Helmholtz coils are less than desirable, since such coils typically suffer from dropouts and field inhomogeneity. Systems making use of toroidal coils are also less than desirable, since such coils have relatively weak field strength.

Perhaps the most serious drawback of prior art systems is that the field generated is incapable of deep penetration into the body of a patient so that the field cannot be concentrated on certain remote diseased area(s) with sufficient amount of energy in a sufficiently short period of time to achieve maximum effectiveness. For this reason PEMF is commonly utilized as adjuvant therapy with other treatment modalities, e.g., medications.

Thus, a need exists for a PEMF apparatus which overcomes the disadvantages of the prior art. The subject invention addresses that need. To that end the apparatus of this invention enables the provision of a pulsed effectively uni-polar electromagnetic field in a focused or concentrated path directed to the exact pathological site (e.g., the “region of interest”) so that it can be treated with high accuracy and with optimum dose whatever the depth of the region of interest.

### SUMMARY OF THE INVENTION

In accordance with one aspect of the invention there is provided apparatus for applying uni-polar magnetic energy to an anatomic region of interest located within the body of a patient. The apparatus basically comprises a housing, a coil, at least one set of focusing magnets, and a source of electrical energy. The coil is a helical electrically conductive member having a central longitudinal axis. The source of electrical energy is arranged when actuated for providing an electrical signal to the coil to produce a magnetic field having a first pole of a first polarity and a second pole of a second polarity, with the longitudinal axis extending through the poles and with lines of flux extending outward from the first pole. The at least one set of focusing magnets comprises at least two focusing magnets each having a first pole and a second pole, the first pole of each of the focusing magnets being of the same polarity as the first pole of the coil and are disposed adjacent the first pole of the coil on opposite sides of the longitudinal axis to distort lines of flux from the first pole and direct such lines of flux in a path extending generally parallel to the longitudinal axis for a substantial distance for impinging the anatomic region of interest in the body of the patient.

Another aspect of this invention entails methods of treating a medical condition of a patient by applying a pulsed electromagnetic field to a region of interest in the body of the patient. The method basically comprises providing apparatus similar to that described above and providing an electrical signal to the coil of the apparatus to produce a pulsed electromagnetic field having a first pole of a first polarity and a second pole of a second polarity, with the longitudinal axis extending through the poles and with lines of flux of the field extending outward from the first pole. The method also entails disposing the first pole of each of the focusing magnets which are of the same polarity as the first pole of the coil adjacent the first pole of the coil on opposite sides of the longitudinal axis to distort the lines of flux from the first pole into a first desired path extending generally parallel to the longitudinal axis. The apparatus is then oriented to direct the lines of flux in the desired path to the region of interest in the body of the patient.

### DESCRIPTION OF THE DRAWING

FIG. 1 is side elevation view, partially in section of one exemplary embodiment of apparatus for providing Uni-polar PEMF therapy constructed in accordance with this invention;

FIG. 2 is an illustration of the use of the apparatus of FIG. 1 for providing Uni-polar PEMF therapy to the brain of a patient to tread a brain disorder or condition;

FIG. 3 is an illustration of the use of the apparatus of FIG. 1 for providing Uni-polar PEMF therapy to the heart of a patient to treat a cardiac condition;

FIG. 4 is an illustration of the use of the apparatus of FIG. 1 for providing Uni-polar PEMF therapy to a representative bone, e.g., the neck of the femur , of a patient to treat osteoporosis;

FIG. 5 is an illustration of the use of the apparatus of FIG. 1 for providing Uni-polar PEMF therapy to a representative bone, e.g., femur head, of a patient to treat avascular necrosis;

FIG. 6 is an illustration of the use of the apparatus of FIG. 1 for providing Uni-polar PEMF therapy to a representative bone, e.g., the femur, of a patient to treat a non-union fracture or a delayed healing after a fracture;

FIG. 7 is an illustration of the use of the apparatus of FIG. 1 for providing Uni-polar PEMF therapy to a representative bone, e.g., the femur, of a patient to treat osteomyelitis (bacterial bone infection);

FIG. 8 is an illustration of the use of the apparatus of FIG. 1 for providing Uni-polar PEMF therapy to an intervertebral disc spine of a patient to treat lumbar and/or cervical pain;

FIG. 9 is an illustration of the use of the apparatus of FIG. 1 for providing Uni-polar PEMF therapy to a representative joint, e.g., the knee, of a patient to treat arthritis; and

FIG. 10 is an illustration of the use of the apparatus of FIG. 1 for providing Uni-polar PEMF therapy to an intervertebral disc spine of a patient to the sacro-coccygeal ligament to treat coccydynia;

### DETAILED DESCRIPTION OF THE PREFERRED EMBODIMENT

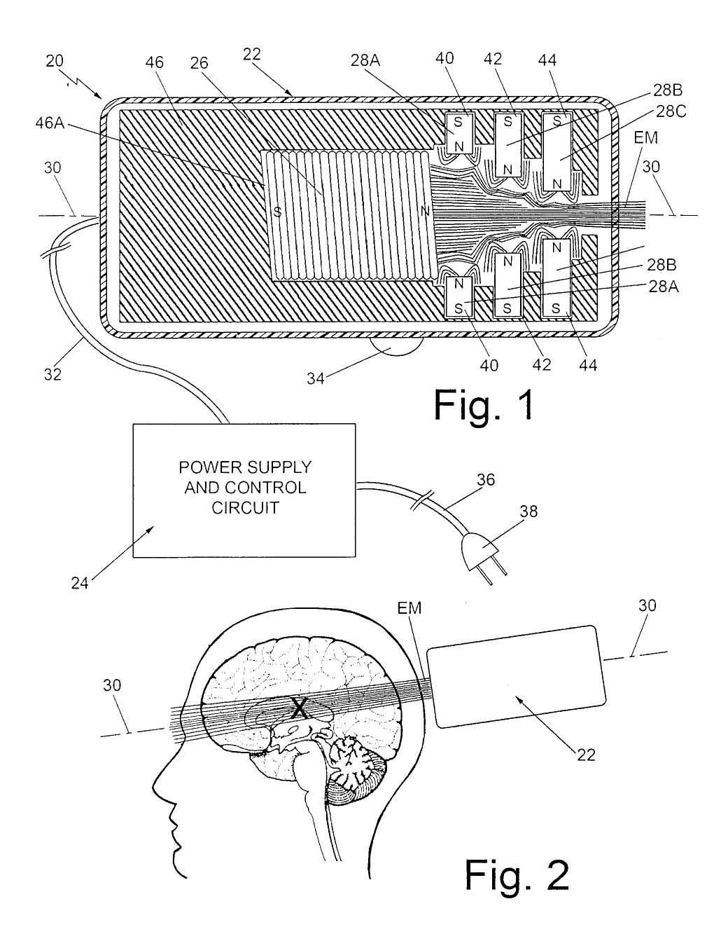

Referring now to the various figures of the drawing wherein like reference t5 characters refer to like parts, there is shown in FIG. 1 at 20 apparatus for producing a unipolar pulsed electromagnetic field which can be directed to great depths into the body of a patient to provide Uni-polar Pulsed Electromagnetic Field (UPEMF) therapy.

In accordance with one preferred exemplary embodiment of this invention, apparatus 20 includes a unit 22 and a unit 24. The unit 22 is arranged to be hand-held or otherwise supported adjacent the portion of the patient's body to receive the UPEMF therapy. The unit 22 basically comprises a housing, an EM field-producing coil 26, a field shaping subassembly 28 and a Mu metal body or shield 46 which are all disposed in the housing. As will be described in considerable detail later the coil 26 serves to produce a pulsed EM field when energized. The field shaping subassembly 28 works in conjunction with the shield 46 to shape the EM field so that it is collimated and effectively uni-polar. The unit 24 is also housed in a housing which contains the electrical circuitry for energizing and controlling operation of the coil 26 in the unit 22. The coil is a helical member formed of any suitable electrically conductive material, e.g., copper, which when energized produces an electromagnetic (EM) field. The coil has a central longitudinal axis 30 extending through it and about which the EM field is centered, with the North pole of the field being designated by the “N” at the rightmost portion of the coil shown in FIG. 1, and with a portion of the lines of flux of the EM field being shown by the lines radiating from the rightmost end of the coil.

The circuitry of the unit 24 for controlling and energizing the coil to produce the EMF field can be of any suitable construction. For example, it can include a regulated power supply, a microcontroller and associated software/firmware and other circuitry, to produce a desired pulsed electric current waveform. That waveform is conveyed to the coil 26 by a cable 32 from the unit 24. The unit 22 includes an ON/OFF switch or button 34 on its housing and which is electrically connected to the cable 32 so that when the button is depressed pulsed current is provided to the coil 26 to excite the coil and thereby produce the pulsed EM field.

The waveform of the current provided to the coil constitutes a series of pulses, which can be a positive, or negative or combinations thereof. Moreover, the pulses can be formed in groups or blasts, with the same or differing intervals of time about 20 minutes per cycle between the individual pulses and/or the pulse blasts. Thus, the coil energizing current waveform can be established to any desired predetermined parameters of a pulse voltage, pulse duration, pulse polarity, number of pulses per unit of time, number of pulses per pulse blast, time duration between pulses in each pulse blast, time duration between pulse blasts, or combinations thereof. In accordance with one exemplary embodiment of this invention the field produced by the coil 24 has a magnetic flux density in the range of 2000 to 6000 gauss and a magnetic field strength in the range of 2000-6000 oersteds.

Electrical power for the unit 24 is provided via a cable 34 and a plug 36 adapted to be connected to a 110V wall outlet (not shown). As mentioned above the unit 24 includes a regulated power supply (which is not shown). That supply serves to bring the line voltage and current to levels suitable for the microprocessor of the unit and for energizing the coil 26. It should be pointed out that instead of having the electrical circuitry in a separate unit from the unit housing the coil, the unit housing the coil can include that circuitry, if desired. Moreover, other sources of electrical power than a wall outlet can be provided. For example, the housing may include one or more batteries to provide electric power to the circuitry of the apparatus to energize the coil 26.

The field shaping subassembly 28 serves to focus, collimate or otherwise shape the magnetic field from a single pole of the coil 26, e.g., the North pole, so that the lines of flux of the field extend generally parallel to the axis of the coil without significant divergence for a significant distance from that pole. This feature enables the apparatus 20 to bring magnetic energy to an anatomic region of interest deep within the body of the patient.

In accordance with a preferred aspect of this invention the field shaping subassembly 28 includes plural sets of focusing magnets disposed adjacent one of the poles of the coil 26. However, a single set of focusing magnets can be used, if desired.

In the exemplary embodiment shown, the field shaping subassembly includes three field shaping sets 28A, 28B and 28C. These set are disposed adjacent the to North pole of the coil 26. Each of the sets includes at least two focusing magnets disposed at equal distances from the central longitudinal axis 30 and equidistantly from each other. It is contemplated that more than two focusing magnets may make up each “pair”, if such is desired. In such a case each of the focusing magnets will be located a predetermined distance from the central longitudinal axis 30 and will be equidistantly spaced from each other. In the exemplary embodiment shown each set 28A, 28B and 28C comprises a pair of focusing magnets disposed on opposite sides of the longitudinal axis 30. In particular, set 28A is made up of a pair of magnets 40, each of which has a North pole (designated by the “N” in FIG. 1). The North pole of each of the focusing magnets of the pair 40 is spaced a predetermined distance from the axis 30 and the poles are directed toward each other such that their lines of flux (shown by the curved lines emanating from their North pole) repulse one another and also repulse the lines of flux of the field produced by the coil 26 and emanating from its North pole to thereby focus those lines of flux to concentrate the lines of flux and direct them parallel to the central longitudinal axis 30.

The set 28B is located adjacent the set 28A and further from the coil's North pole than the set 28A. The set 28B is made up of a pair of magnets 42, each of which has a North pole (designated by the “N” in FIG. 1). The North pole of each of the focusing magnets of the pair 42 is spaced a predetermined distance from the axis 30 and those poles are directed toward each other. The spacing between the North poles of the focusing magnets 42 is less than the spacing between the North poles of the focusing magnets 40 since the magnetic field produced by the coil 26 in the vicinity of the magnets 42 will have naturally diverged from the central longitudinal axis but for the action of the focusing coils 42. In particular, since the North poles of the magnets 42 are closer to the central longitudinal axis 30, the fields produced by them will have a equalizing repulsive effect on the lines of flux of the magnetic field produced by the coil 26 to concentrate the those lines of flux and direct them parallel to the central longitudinal axis 30 in a manner similar to that achieved by the focusing magnets 40.

In a similar manner, the set 28C is located adjacent the set 28B and further from the coil's North pole than the set 28B. The set 28C is made up of a pair of magnets 44, each of which has a North pole (designated by the “N” in FIG. 1). The North pole of each of the focusing magnets of the pair 44 is spaced a predetermined distance from the axis 30 and those poles are directed toward each other. The spacing between the North poles of the focusing magnets is less than the spacing between the North poles of the focusing magnets 42 since the magnetic field produced by the coil 26 in the vicinity of the magnets 44 will have naturally diverged even further from the central longitudinal axis but for the action of the focusing coils 44. Thus, the North poles of the magnets 42 are closer to the central longitudinal axis 30 so that the fields produced by them will have an equalizing repulsive effect on the lines of flux of the magnetic field produced by the coil to concentrate the lines of flux and direct them parallel to the central longitudinal axis 30 in a manner similar to that achieved by the focusing coils 40 and 42.

As a result of the action of the focusing magnets the lines of flux of the field produced by the coil 26 will be focused or collimated so that they are concentrated and extend generally parallel to the central longitudinal axis 30 for a substantial distance beyond the end of the housing of the unit 22 as shown in FIG. 1. It should be noted that while the lines of flux produced by the coil 26 do curve back to the South pole of the coil, they are shaped and confined by the Mu metal shield 46. In particular, as can be seen in FIG. 1, the Mu metal shield 46 encompasses or surrounds virtually all the internal components of the unit 22, i.e., it surrounds all of the internal structures of the unit 22 except for the narrow column centered on the central longitudinal axis 30 and projecting outward from the North pole of the coil 26 and for the North poles of each of the focusing magnets 40, 42, and 44. The shield 46 works in cooperation with the focusing magnets so that the EM field emanating from the unit 22 is effectively uni-polar. In particular, the portion of the EM field which emanates from the North pole end of the unit 22 is shaped by the action of the focusing magnets and the Mu metal body within the housing so that the portion of the field that projects out of the North pole end of the housing is condensed or collimated. This collimated uni-polar EM field can be directed into the patient's body to effectively reach any region of interest to be treated irrespective of the depth of that region of interest. In the exemplary embodiment shown in FIG. 1, the unipolar EM field is of North polarity, i.e., it consists of the concentrated lines of flux from the coil's North pole.

In order to shape the EM field so that it is effectively collimated as it passes through the focusing subassembly 28, notwithstanding the tendency of the field's lines of flux to want to spread out and loop back to the south pole of the coil, the magnets 40 making up the set 28A should be stronger than the magnets 42 making up the set 28B, which in turn should be stronger than the magnets 44 making up the set 28A. This has an equalization effect on the flux lines of the EM field thus effectively concentrating them and causing them to be more linear and not diverging greatly from the central longitudinal axis 30. Moreover, while the focusing magnets of the sets 28A, 28B and 28C have been described as being permanent magnets, that is merely exemplary. Thus, any or all of the focusing magnets may themselves be electromagnets. If electromagnets are used as focusing magnets, they need not be pulsed.

If desired, the Mu metal body 46 may include a hollow space or chamber 46A located adjacent the South pole of the coil 26. For many therapies it is desired to utilize the North pole as the treating modality, since the North pole seems to provide better physiological effects on human cells. Thus in the exemplary embodiment the unit 22 is arranged to have the focusing magnets adjacent the North pole of the coil and with the South pole being shielded by the Mu metal body 46. For other applications, the South pole of the apparatus 20 may be utilized. In such a case the focusing magnets will be located adjacent the South pole of the coil 26, with the South pole of each of the focusing magnet pairs facing the central longitudinal axis 30 and with the Mu metal body 46 surrounding the North Pole of the coil and the North poles of the focusing magnets.

It should be pointed out at this juncture that the focusing magnets making up the various sets need not to be spaced away from the central axis 30 as shown in FIG. 1. Thus, they can all be the same distance from that axis or any or all could be at different distances, providing that that the strength of the field produced by the magnets of each set is sufficient to focus the coil's magnetic field in a manner as described above. It should also be noted that the coil 26 may, if desired, include a ferromagnetic core instead of an air-core.

Use of the apparatus 20 for providing various UPEMF therapies in accordance with this invention will now be considered. For example, FIG. 2 is an illustration of the exemplary apparatus 20 of FIG. 1 shown in the process of treating a brain disorder or condition, such as but not limited to some brain pathology selected from the group comprising Alzheimer's disease, Parkinson's disease, dementia, migraine and senile atrophy of the brain. To that end, as can be seen, the apparatus is disposed adjacent the skin of the patient at any desired position adjacent the patient's head and oriented to direct the “unipolar” EMF to the brain 50 at the situs of the pathology (shown by the X in FIG. 2) to be treated. In this exemplary case, the situs of the pathology X is in the mid-brain area. When the unit 22 of the apparatus 20 is in the desired position and orientation the ON/OFF switch or button 34 can be pressed to turn the apparatus on and thereby produce the EMF field directed along the longitudinal central axis 30. Since the EM field is concentrated and focused and centered on the axis 30 it can thus penetrate through the skin and underlying tissue and bone of the skull to the situs of the pathology.

As is well known, many brain diseases or conditions are caused by damage (degenerative changes) that occurs in a specific site in brain substance which will lead to a decreased local energy production in this area with subsequent reduction in electro-chemical transmission. Thus, the higher amount of energy carried by the field into the region of interest without significant attenuation by the intervening tissue/bone should bring better results and accelerate the healing process as compared to the prior art so it can be used as an important core therapy, not only as an adjuvant therapy.

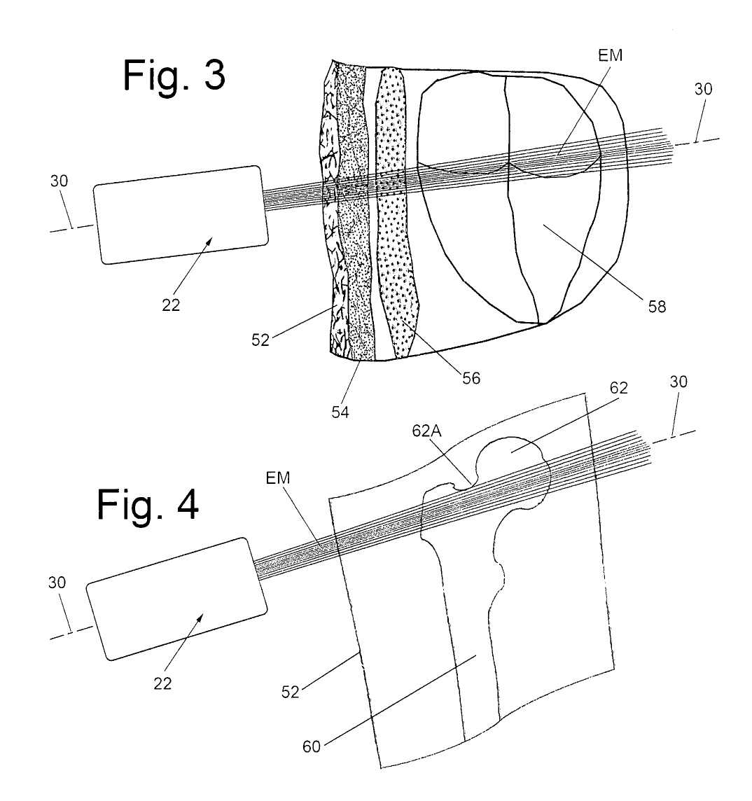

Use of the apparatus 20 for treating chronic coronary diseases, such as coronary artery disease (CAD), cardio-myopathy, a previous myocardial infarction (death and fibrosis of part of the heart muscle) in those patients who have a high incidence of repeated infarction, is shown in FIG. 3. As will be appreciated by those skilled in the art a blockage or even narrowing of certain blood vessels to certain part(s) of cardiac muscle will decrease oxygen and nutrition to that area with subsequent a decrease of local energy production. Thus the affected area becomes weak and incapable of performing properly. The exposure of that cardiac tissue to the pulsed electromagnetic field provided by the subject apparatus 20 should compensate for that lack of the energy production, by providing additional energy to the cardiac tissue resulting from the collision of the pulsed electromagnetic field with that cardiac tissue. The apparatus of the subject invention is particularly suitable for this type of application since the heart is a deep seated structure lying behind the skin and subcutaneous tissues, chest wall muscles and ribs. In obese persons (a quite common type of cardiac patient) location of the region of interest in the heart is likely to be even more remote since such patients have excess fat intervening between the skin and the region of interest.

To treat the cardiac condition the apparatus is disposed adjacent the skin of the patient at any desired position on the patient's torso to direct the “unipolar” EMF to the heart at the situs of the region to be treated. In this exemplary case, the skin and underlying tissue of the patient is designated by the reference number 52, the patient's chest muscles and ribs are designated by the reference number 54, and the pericardial fat is designated by the reference number 56. When the apparatus 20 is in the desired position and orientation the ON/OFF switch can be pressed to turn the apparatus on and thereby produce the UPEMF field directed along the longitudinal central axis 30. Since that field is concentrated, focused and centered on the axis 30 it can thus penetrate through the skin and underlying tissue, bone and pericardial fat to the region of interest in the heart 58.

Use of the apparatus 20 for providing PEMF therapy to a representative bone, e.g., the neck of the head of the femur, of a patient to treat osteoporosis is shown in FIG. 4. As is known osteoporosis is a disease affecting the elderly population and frequently results in bone fracture with mild trauma. Osteoporosis generally affects all bones of the body but certain bones are more critical than other if affected. For example, the bones of neck of femur, spine, and pelvic bones if affected by osteoporosis can lead to significant injury, e.g., fracture and concomitant trauma. In case of affection of the neck of the femur, such as shown in FIG. 4, its fracture needs urgent surgical intervention otherwise the patient may die. The neck of femur is a deep structure and surrounded by many muscles. Since the field produced by the apparatus can penetrate deeper tissues with higher amount of energy than ordinary PEMF it can be very important to treat osteoporosis. Therefore, use of this invention provides the possibility of preventing fracture of the neck of the femur and subsequent surgical fixation. To that end, the unit 22 of the apparatus 20 is disposed adjacent the skin of the patient and oriented to direct the “unipolar” EMF to the femur 60 at the situs of the osteoporosis at the neck 62A of the femur. When the apparatus 20 is in the desired and orientation position the ON/OFF button can be pressed to turn the apparatus on and thereby produced the EMF field. Since that field is concentrated and focused it can thus penetrate through the skin, subcutaneous tissue and intervening thigh muscles to the region of interest at the femoral neck 62A reverse or at least impede the progress of the osteoporosis thereat.

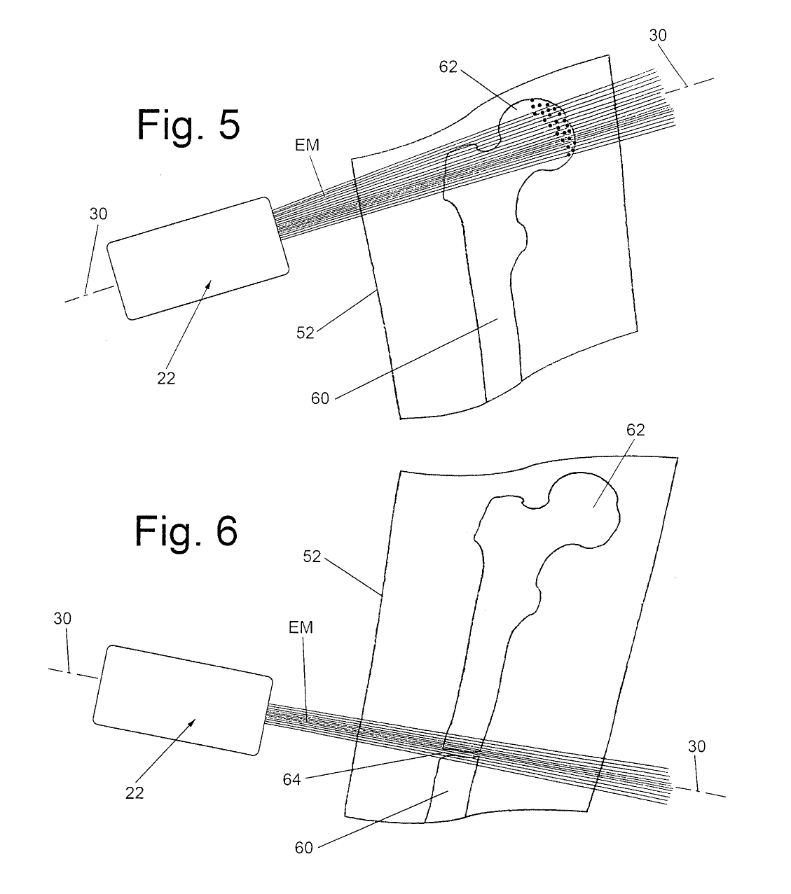

Use of the apparatus 20 for providing PEMF therapy to a representative bone, e.g., the head of the femur, of a patient to treat avascular necrosis (AVN) is shown in FIG. 5. As is known AVN is bone death and commonly results from a chronic deficiency of blood supply to certain part of bone. Lack of blood supply to certain part of bone reduces the maintenance of bone turnover with subsequent degenerative changes in that area. The accumulative effect of the lack of an adequate blood supply to certain bone may end by death of that bone because of lack of local energy production. PEMF offers a good response in the treatment of this condition because collision of the pulsed electromagnetic field and that bone generate energy that can compensate for the lack of energy resulting from an insufficient blood supply. Thus, PEMF offers a modality which may avoid the necessity of surgical intervention to treat the AVN. However, since the affected bone is usually in a deep seated position because it is supplied by end organ artery. e.g., head of the femur and the inner femoral condyle of knee it is difficult to treat with prior art PEMF apparatus. The apparatus of subject invention, with its ability to reach deep seated structures enables its viable use to treat AVN. Thus, the apparatus 20 can be used in a similar manner to that described above with respect to FIG. 4 to treat AVN as shown in FIG. 5. To that end, the unit 22 is operated in a similar manner to that described with reference to FIG. 4 to deliver the concentrated EM field to the situs of the avascular necrosis which is shown by the dotted area in FIG. 5.

Use of the apparatus 20 for providing PEMF therapy to a representative bone, e.g., the femur, of a patient to treat a non-union fracture or a delayed healing after a fracture is shown in FIG. 6. Thus, as can be seen the apparatus is disposed adjacent the skin 52 of the patient and oriented to direct the “unipolar” EMF to the femur 60 at the situs of the fracture 64. When the apparatus 20 is in the desired position and orientation the ON/OFF button can be pressed to turn the apparatus on and thereby produced the EMF field. Since that field is concentrated and focused it can thus penetrate through the skin and intervening thigh muscles to the region surrounding the fracture 64, e.g., the region of interest along the bone portions contiguous with the fracture to facilitate its healing. The application of a pulsed magnetic field to treat bone fracture with delayed union and non-union should provide promising results by accelerating the healing process by the energy of unipolar PEMF that is able to activate stem cells that enhance new bone formation so it can be used as an important core therapy not only as an adjuvant therapy.

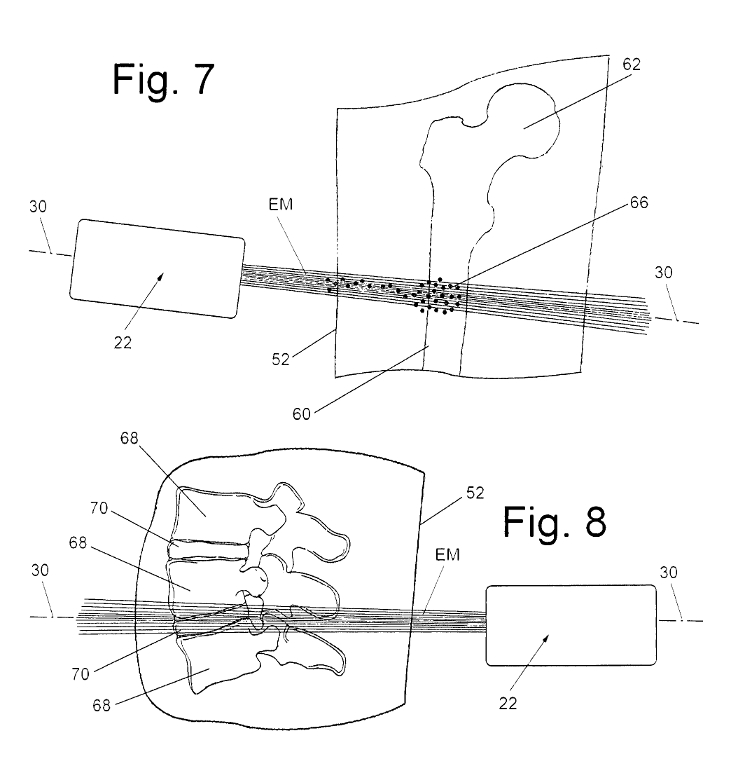

FIG. 7 is an illustration of the apparatus 20 shown in FIG. 1 in the process of treating osteomyelitis in accordance with another exemplary method of this invention. Chronic osteomyelitis (septic bone infection) is catastrophic disease that is very difficult to treat and a full cure is sometimes impossible. It needs a long term course of treatment, sometimes over years with repeated surgical intervention in many cases. With this disease, bacteria live in devitalized bone and hence are remote from the immune system. PEMF should prove beneficial for this condition by producing local heat at the situs of the infection. This heat, in turn, should increase the blood supply and raise the immunity by increasing the number of immune cells (macrophages and lymphocytes) in the affected devitalized bone. Thus, FIG. 7 shows the unit 22 of the apparatus 20 brought into position closely adjacent and oriented toward the situs of an osteomyelitis infection 66 within the femur 60. The apparatus is then operated in a similar manner to that described above to deliver the concentrated EM field to the infection.

FIG. 8 is an illustration of the exemplary apparatus 20 of FIG. 1 in the process of treating chronic low back pain by directing the pulsed electromagnetic field to the facet joints, the back of the vertebral bodies 68 and intervening discs 70 of the spine in accordance with another exemplary method of this invention. Chronic low back pain is a group of diseases that affects a large number of the population (e.g., approximately 25 million in the United States), especially obese elderly women. One common cause of low back pain is that the intervertebral disc herniates and compresses the spinal nerve causing severe neurological pain. In case of the neck, the pain is referred to shoulder. In case of lumbar spine the pain is radiated or referred to the legs. The mechanism of pain is compression of the spinal nerve by the protruded substance of the disc with subsequent reduction of blood supply of affected nerve resulting in reduction of local energy production in the affected nerves. The treatment with PEMF directly supplies the energy to these compressed nerves and thus can reverse the whole pathological process. Therefore, the pain can gradually subside. The electromagnetic field produced by the apparatus of the subject invention is expected to be more effective than conventionally produced PEMF because it has more energy and can penetrate deeper tissues. In this regard, as shown in FIG. 8 the pulsed electromagnetic field EM produced by the coil 26 of the unit 22 can traverse the skin, subcutaneous tissues, long spinous process, facet joint, and intervertebral canal to reach to the protruding disc 70. Conventional devices for providing PEMF hardly can penetrate all these tissues/structures.

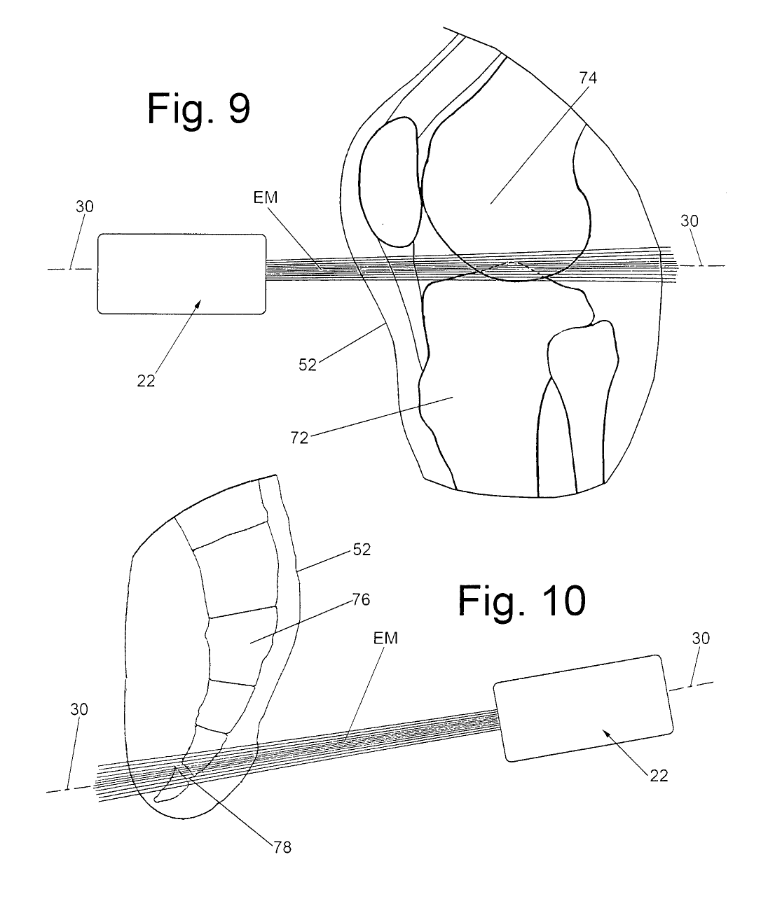

FIG. 9 is an illustration of the apparatus shown in FIG. 1 in the process of treating joint arthritis in accordance with another exemplary method of this invention. The exemplary arthritic joint shown in this example is the tibia-femur joint of the knee. As is known, osteoarthritic joint diseases are degenerative joint diseases which break down the articular cartilage which become thin, eroded, and sometimes ulcerated with reduction of lubricant materials (e.g., synovial fluids) leading to painful movements. The result of this condition typically involves protective reduction of the movements of the patient with subsequent muscle weakness. Consequently, there will be joint instability resulting in further damage of articular cartilage and the patient enters in a vicious cycle. Treatment with PEMF offers a way to improve the condition because it breaks the cycle at all pathological steps. In particular, exposure of the joint to PEMF provides that joint with external energy via the introduced pulsating electromagnetic field, with a subsequent increase in the secretion of synovial fluid, all of which facilitates joint movement with less pain. The introduced pulsating electromagnetic field also helps increase the blood supply to muscles surrounding the joint, thereby reducing joint instability thus protecting the articular cartilage. Moreover, recent studies show that PEMF is able to stimulate chondrocyte to help in repairing of the damaged cartilage. Thus, as shown in FIG. 9, the unit 22 of the apparatus 20 is positioned and oriented so that its longitudinal central axis 30 is directed to the joint between the tibia 72 and the femur 74. The ON/OFF switch is then depressed to energize the coil and thus produce the concentrated EM field and direct it to that joint.

FIG. 10 is an illustration of the apparatus 20 of FIG. 1 shown in the process of treating coccydynia in accordance with another exemplary method of this invention. Coccydynia is disease is common in women after giving birth to have chronic pain at the lower end of the vertebral column 76. It is considered to be due to injury of sacro-coccygeal ligament during delivery. It can occur in males, but such occurrences are rare. Coccydynia is resistant to all methods of treatment even surgical excision may not be helpful. Treatment with the apparatus 20 of this invention can result in rapid healing in many cases and should be due to its higher capacity of penetration to supply energy to the deep sacro-coccygeal ligament. The pulsed electromagnetic field EM has to penetrate the skin, subcutaneous tissues, thick muscle layer, the sacrum and coccyx bone, to reach the sacro-coccygeal ligament 78 at the sacro-coccygeal joint. Thus, the treatment of this condition with conventional PEMF apparatus is problematic. In contradistinction, the apparatus 20 of this invention offers a significant modality for treating coccydynia irrespective of the depth of the sacro-coccygeal joint from the skin. To that end, as can be seen in FIG. 10 the apparatus 20 is positioned and oriented so that the concentrated electromagnetic field EM is directed to the sacro-coccygeal ligament 78 at the sacro-coccygeal joint and can pass through the intervening anatomic structures.

As should be appreciated by those skilled in the art from the foregoing the subject invention offers numerous advantages over the conventional apparatus for providing PEMF. In particular, the subject invention by-passes the skin barrier and allows penetration to deeper tissues/structures, e.g., three inches or greater. In fact an apparatus constructed in accordance with this invention and their methods of use can apply a pulsed electromagnetic field at virtually any depth within the body of the patient to apply maximum energy to the region of interest. Moreover, the narrow or somewhat collimated field produced by this invention results in the delivery of the pulsed electromagnetic field to a reduced size surface or volume so that it can absorb a great part of applied energy, as opposed to prior art PEMF systems which are characterised by dissipated or widely divergent fields. In short, the straight and narrow electro-magnetic field produced by the subject invention allows it to travel deeper to effectively treat the region of interest. Although the lines of flux of the EM field do diverge from each other they are still not widely separated from each other even after they exit from human body. Recent studies show that there is a relation between divergence of the electromagnetic field and its strength. The stronger the electromagnetic field, the less the divergence. Therefore, divergence is minimal inside the human body but after its exit from the body, it loses greater part of its strength (the part that absorbed by human tissues). The remaining part becomes weak and easily divergent.

It should be noted that the particular components and their arrangements as discussed above and as shown in the drawings are merely exemplary. Thus, other components can be used in lieu of those disclosed. For example, the shield 46 may be formed of some material having similar EM field modifying effects as Mu metal. Without further elaboration the foregoing will so fully illustrate our invention that others may, by applying current or future knowledge, adopt the same for use under various conditions of service.

I claim:

1. Apparatus for applying uni-polar magnetic energy to an anatomic region of interest located within the body of a patient, said apparatus comprising a housing, a coil, at least one set of focusing magnets, and a source of electrical energy, said coil being an electrically conductive helical member having a central longitudinal axis, said source of electrical energy being arranged when actuated for providing an electrical signal to said coil to produce a magnetic field having a first pole of a first polarity and a second pole of a second polarity, with said longitudinal axis extending through said poles and with lines of flux extending outward from said first pole, said at least one set of focusing magnets comprising at least two focusing magnets each having a first pole and a second pole, said first pole of each of said focusing magnets being of the same polarity as said first pole of said coil and being disposed adjacent said first pole of said coil on opposite sides of said longitudinal axis to distort lines of flux from said first pole and direct such lines of flux in a path extending generally parallel to said longitudinal axis for a substantial distance for impinging the anatomic region of interest in the body of the patient.

2. The apparatus of claim 1 wherein said apparatus additionally comprises a Mu metal body surrounding said second pole.

3. The apparatus of claim 1 wherein said apparatus additionally comprises a Mu metal body surrounding said second poles of said focusing magnets.

4. The apparatus of claim 2 wherein said apparatus additionally comprises a Mu metal body surrounding said second poles of said focusing magnets.

5. The apparatus of claim 1 wherein each of said focusing magnets is a permanent magnet.

6. The apparatus of claim 1 wherein each of said focusing magnets is an electromagnet.

7. The apparatus of claim 1 wherein said apparatus includes at least two sets of focusing magnets, each set of focusing magnets comprising at least two focusing magnets each having a first pole and a second pole, wherein a first set of said focusing magnets is located closer to said first pole of said coil than a second set of said focusing magnets, said first poles of said at least two focusing magnets of said first set being disposed on opposite sides of said longitudinal axis a first predetermined distance from said longitudinal axis, said first pole of said at least two focusing magnets of said second set being disposed on opposite sides of said longitudinal axis a second predetermined distance from said longitudinal axis, said second predetermined distance being less than said first predetermined distance.

8. The apparatus of claim 7 wherein each of said focusing magnets of said first set is stronger than each of said focusing magnets of said second set.

9. The apparatus of claim 2 wherein said apparatus includes at least two sets of focusing magnets, each pair of focusing magnets comprising at least two focusing magnets each having a first pole and a second pole, wherein a first set of said focusing magnets is located closer to said first pole of said coil than a second set of said focusing magnets, said first poles of said at least two focusing magnets of said first set being disposed on opposite sides of said longitudinal axis a first predetermined distance from said longitudinal axis, said first pole of said at least two focusing magnets of said second set being disposed on opposite sides of said longitudinal axis a second predetermined distance from said longitudinal axis, said second predetermined distance being less than said first predetermined distance.

10. The apparatus of claim 9 wherein each of said focusing magnets of said first set is stronger than each of said focusing magnets of said second set.

11. The apparatus of claim 1 wherein said source of electrical energy comprises an electrical circuit coupled to said coil which when actuated causes said coil to produce a pulsed electromagnetic field.

12. The apparatus of claim 2 wherein said source of electrical energy comprises an electrical circuit coupled to said coil which when actuated causes said coil to produce a pulsed electromagnetic field.

13. A method of treating medical condition of a patient by applying a pulsed uni-polar electromagnetic field to a region of interest in the body of the patient, said method comprising:
a) providing apparatus comprising a housing, a coil, at least one set of focusing magnets, and a source of electrical energy, said coil being an electrically conductive helical member having a central longitudinal axis, said at least one set of focusing magnets comprising at least two focusing magnets each having a first pole of a first polarity and a second pole of a second polarity;
b) providing an electrical signal to said coil to produce a pulsed electromagnetic field having a first pole of a first polarity and a second pole of a second polarity, with said longitudinal axis extending through said poles and with lines of flux of said field extending outward from said first pole; and
c) disposing said first pole of each of said focusing magnets which are of the same polarity as said first pole of said coil adjacent said first pole of said coil on opposite sides of said longitudinal axis to distort said lines of flux from said first pole into a first desired path extending generally parallel to said longitudinal axis; and
d) orienting said apparatus to direct said lines of flux in said desired path to the region of interest in the body of the patient.

14. The method of claim 13 wherein said medical condition comprises brain pathology, and wherein the region of interest is in the brain of the patient.

15. The method of claim 14 wherein said brain pathology is selected from the group comprising Alzheimer's disease, Parkinson's disease, dementia, migraine and senile atrophy of the brain.

16. The method of claim 13 wherein said medical condition comprises cardiac disease, and wherein the region of interest is in the heart of the patient.

17. The method of claim 16 wherein said cardiac disease is selected from the group comprising coronary artery disease, cardiomyopathy, and prior myocardial infarction.

18. The method of claim 13 wherein said medical condition comprises an orthopedic condition, and wherein the region of interest is in the bone of the patient.

19. The method of claim 18 wherein said orthopedic condition is selected from the group comprising osteoporosis, avascular necrosis, non-union or delay bone healing after fracture, osteomyelitis, intervertebral disc herniation or compression, arthritis, and coccydynia.
